(19) United States
(12) Patent Application Publication
(10) Pub. No.: US 2014/0127161 A1
(43) Pub. Date: May 8, 2014

(54) TRICHODERMA STRAINS, METHOD FOR PREPARATION THEREOF AND THEIR USE AS BIOCIDES

(71) Applicant: INSTYTUT BIOCHEMII I BIOFIZYKI PAN, WARSZAWA (PL)

(72) Inventors: Joanna S. Kruszewska, Warszawa (PL); Urszula Perlinska-Lenart, Warszawa (PL); Sebastian Pilsyk, Warszawa (PL); Wioletta Gorka-Niec, Chyliczki (PL); Patrycja Zembek, Minsk Mazowiecki (PL); Sebastian Graczyk, Brody (PL); Grazyna Palamarczyk, Warszawa (PL)

(73) Assignee: INSTYTUT BIOCHEMII I BIOFIZYKI PAN, WARSZAWA (PL)

(21) Appl. No.: 14/067,597

(22) Filed: Oct. 30, 2013

(30) Foreign Application Priority Data
Nov. 2, 2012 (PL) P.401450

(51) Int. Cl. A01N 63/04; C12N 15/80

(52) U.S. Cl. CPC A01N 63/04; C12N 15/80
USPC 424/93.2; 435/254.6; 435/471

(57) ABSTRACT

The invention provides novel strains of Trichoderma spp. showing increased biocidal activity and the method for preparation thereof and use thereof as a biocide. The invention especially provides a new strain of Trichoderma atroviride having high antifungal activity, in particular to be used in agriculture as a bio-fungicide. The invention also includes a new antimicrobial composition containing the new Trichoderma strains, and a new composition enhancing plant growth containing the new strains.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 480320_401_SEQUENCE_LISTING.txt. The text file is 4.9 KB, was created on Oct. 29, 2013, and is being submitted electronically via EFS-Web.

### BACKGROUND

The present invention provides novel strains of Trichoderma spp having biocidal activity, and a process for preparing such strains and their use as biocide, especially as fungicide. In particular, the strains of the invention are used in agriculture as biofungicides. The invention also relates to a biocidal composition for biological control of unwanted organisms. Fungi of the Trichoderma genus have natural antifungal properties. Many of them could parasitize on fungi pathogenic to plants. As a result of genetic modification described below we obtained the new strains of Trichoderma with unique antifungal properties. There are also fungi from Trichoderma genus known from their natural insecticidal properties. For example, genetically engineered strains of Trichoderma asperellum having exceptional insecticidal properties are also included in the invention.

Trichoderma constitutively secretes a small amount of hydrolytic enzymes. If pathogenic fungus or insect having carbohydrate polymers in their cell wall or cuticle, respectively, appear near Trichoderma the carbohydrates could become a substrate for hydrolases secreted by Trichoderma. Furthermore, it is believed that Trichoderma constitutively secretes a small amount of chitinases which encountering cell wall of fungi or insects cuticle cutting out monosaccharides from chitin which gives a signal to increase expression of hydrolases such as chitinases, glucanases and proteases, and the type of secreted hydrolytic enzymes depends on the attacked object. In consequence, cell wall of foreign fungus or insect is hydrolyzed and the organisms are destructed. This way Trichoderma attacks plant pathogenic fungi or insects and, at the same time remains in symbiosis with plant.

It is known that Trichoderma is used to control pathogenic fungi which are destructive for agricultural production and that the biocontrol properties could be enhanced by overexpression of genes coding for various hydrolytic enzymes. The Polish patent application no P393021 describes how to activate biocontrol activity of Trichoderma, especially T. atroviride strain by activating the O-glycosylation process, seemingly unrelated to the protein secretion. The solution described in P393021 based on the relationship between the process of O-linked glycosylation and secretion of hydrolytic enzymes (Kruszewska et al 1999). Hydrolases secreted by Trichoderma constitute an important element of Trichoderma attack on fungi pathogenic to plants. Hydrolases are glycoproteins and their efficient glycosylation affects their production and secretion. By increasing the activity of dolichyl phosphate mannose synthase (DPM), a key enzyme of the O-glycosylation process, faster glycosylation of hydrolases was achieved and thereby increased secretion of theses enzymes. These resulted in more active digestion of the cell wall of pathogenic fungi giving T. atroviride with better biocontrol properties.

Many crop plants and flowers are damaged by fungal or other infections every year. These infections are controled using chemical fungicides and pesticides, which are introduced into the environment. This can be avoided or reduced by the present invention.

Infections by Fusarium, Pythium, Botritis cinerea, Phytophthora infestans are one of the common infections of crops. These fungi attack vegetables, fruits, ornamental plants, trees and shrubs. In addition to the chemicals biocontrol agents could be used for plant protection. It was found that simultaneous application of Trichoderma together with conventional fungicides can limit the amount of the latter by more than 20%.

Thus, there is still a need to develop new, more effective methods of biological control of crops using bio-fungicides which are harmless to the environment.

Furthermore, it was surprisingly found that by using the solution according to the invention not only a new more effective biocontrol method was developed but also a better growth of plants was achieved. Surprisingly, it was also found that Trichoderma asperellum, modified according to the invention, can be used as an insecticide and also can improve the growth of plants.

According to the invention we present a new method for obtaining strains of Trichoderma spp. with improved antifungal and biocontrol properties. This method is based on the increase in the synthesis of the mevalonate pathway products.

The subject of the invention is a mutated strain of Trichoderma spp. containing a recombinant DNA including ERG20 gene encoding farnesyl pyrophosphate synthase. Preferably, Trichoderma is Trichoderma atroviride. Preferably, the gene encoding farnesyl pyrophosphate synthase originates from Saccharomyces cerevisiae, and the gene encoding farnesyl pyrophosphate synthase codes for protein of SEQ ID NO:2. Preferably, the gene encoding farnesyl pyrophosphate synthase comprises the nucleotide sequence as set forth in SEQ ID NO:1. In certain embodiments, the farnesyl pyrophosphate synthase comprises an amino acid sequence that is at least 50%, at least 55%, at least 60%, at least 65%, at least 70%, at least 75%, at least 80%, at least 85%, at least 90%, at least 95%, or 100% identical with SEQ ID NO:2. Preferably, the ERG20 gene from S. cerevisiae is integrated into the genome of Trichoderma. Preferably, the ERG20 gene is integrated into the genome and overexpressed. Methods to determine sequence identity can be applied from publicly available computer programs. Computer program methods to determine identity between two sequences include, for example, BLASTP, BLASTN (Altschul, S.F. et al., J. Mol. Biol. 215: 403-410 (1990), and FASTA (Pearson and Lipman Proc. Natl. Acad. Sci. USA 85; 2444-2448 (1988). The BLAST family of programs is publicly available from NCBI and other sources (BLAST Manual, Altschul, S., et al., NCBI NLM NIH Bethesda, Md.

The invention also provides a method for preparing the transgenic Trichoderma strain according to the invention, in which the DNA encoding farnesyl pyrophosphate synthase is isolated, then Trichoderma strain is transformed with this gene and overexpression of farnesyl pyrophosphate synthase in the resulting transgenic strain is obtained.

Preferably, the method leads to the integration of the recombinant DNA coding for farnesyl pyrophosphate synthase into the genome of Trichoderma.

The invention also provides a method of controlling undesired organisms using Trichoderma strain with overexpression of the DNA encoding farnesyl pyrophosphate synthase. An effective amount of the Trichoderma strain may be used to contact the undesired organisms or substances (e.g., soil or plant parts or materials) suspected to contain undesired organisms. Preferably, in the method according to the invention, Trichoderma strain is used in which overexpression of the homologous DNA coding for farnesyl pyrophosphate synthase from S. cerevisiae occurs. Preferably, fungal diseases of plants and crops are combated (treated, inhibited or prevented). Particularly preferably, Pythium ultimum is combated.

The invention also provides a method for increasing plant growth and/or crop yield, when Trichoderma strain carrying overexpressed DNA encoding farnesyl pyrophosphate synthase according to the invention is used. An effective amount of the Trichoderma strain may be applied to plants/crops, portions of plants/crops (e.g., seeds, stems, or leaves), or environment (e.g., soil) surrounding plants/crops or portions thereof.

The invention also includes a biocidal composition which comprises a strain of Trichoderma which according to the invention carries overexpression of DNA coding for farnesyl pyrophosphate synthase. Furthermore, the invention relates to a composition for increasing crops yield including Trichoderma strain that has been provided herein, in which DNA encoding farnesyl pyrophosphate synthase is overexpressed.

### BRIEF DESCRIPTION OF THE DRAWINGS

FIG. 1 Mevalonate pathway

FIG. 2 Influence of ERG20 overexpression on the activity of farnesyl pyrophosphate synthase (FPP). P1—control T. atroviride; 20, 59, 104, 125, 133—transformants SGUL20/10, SGUL59/10, SGUL104/10, SGUL125/10 and SGUL133/10, respectively

FIG. 3A Growth of the Trichoderma transformants on plates with minimal medium containing carboxymethylcellulose as a carbon source. P1—control T. atroviride; 20, 59, 104, 125, 133—transformants SGUL20/10, SGUL59/10, SGUL104/10, SGUL125/10 and SGUL133/10, respectively.

FIG. 3B A halo surrounding colonies of Trichoderma growing on minimal medium with carboxymethylcellulose. P1—control T. atroviride; 20, 59, 104, 125, 133—transformants SGUL20/10, SGUL59/10, SGUL104/10, SGUL125/10 and SGUL133/10, respectively

FIG. 4A Cellulolytic activity measured in the culture medium during cultivation of Trichoderma strains. Concentration of reducing sugars cleaved out from substrate (carboxymethylcellulose) was measured in cultivation medium from SGUL20/10, SGUL59/10, SGUL104/10, SGUL125/10 and SGUL133/10 transformants and the parental strain P1. P1—control T. atroviride; 20, 59, 104, 125, 133—transformants SGUL20/10, SGUL59/10, SGUL104/10, SGUL125/10 and SGUL133/10, respectively

FIG. 4B N-acetyl-β-D-glucosaminidase activity measured in the culture medium during cultivation of Trichoderma strains. Concentration of p-nitrophenol cleaved out from the substrate (p-nitrophenyl-β-D-N-acetylglucosaminide) was measured in cultivation medium from SGUL20/10, SGUL59/10, SGUL104/10, SGUL125/10 and SGUL133/10 and control P1 strain. P1—control T. atroviride; 20, 59, 104, 125, 133—transformants SGUL20/10, SGUL59/10, SGUL104/10, SGUL125/10 and SGUL133/10, respectively

FIG. 5 Protein secretion measured in the culture medium during cultivation of Trichoderma strains in the medium containing lactose as a carbon source. P1—control T. atroviride; 20, 59, 104, 125, 133—transformants SGUL20/10, SGUL59/10, SGUL104/10, SGUL125/10 and SGUL133/10, respectively

FIG. 6 Growth of Pythium ultimum colonies on plates pretreated with Trichoderma strains SGUL20/10, SGUL59/10, SGUL104/10, SGUL125/10, SGUL133/10 and the parental strain were cultivated on minimal medium plates covered with cellophane. Pythium ultimum was inoculated on the pretreated plates. As a control P. ultimum was cultivated on a non-pretreated plates.

FIG. 7 Growth of Rhizocthonia solani in the atmosphere of volatiles liberated by Trichoderma strains. Rhizocthonia—R. solani cultivated without Trichoderma. P1—R. solani cultivated in the atmosphere of volatiles liberated by the control P1 strain. 20, 59, 104, 125, 133—R. solani cultivated in the atmosphere of volatiles liberated by the transformants SGUL20/10, SGUL59/10, SGUL104/10, SGUL125/10 and SGUL133/10, respectively.

FIG. 8 Protective effect of Trichoderma on bean seedlings growing in P. ultimum infected soil. Average size of seedlings measured as seedling height (cm) was analyzed after ten days. Positive control (control)—growth of beans in the sterile soil; Pythium—negative control growth of beans in the soil infected by P. ultimum; P1, 20, 59, 104, 125, 133—growth of beans in soil infected by P. ultimum and Trichoderma (gray bars) or only Trichoderma (black bars).

FIG. 9 Protective effect of Trichoderma on bean germination in P. ultimum infected soil. Efficiency of germination presented as percentage of seeds sown was analyzed after ten days. Positive control (control)—germination of beans in the sterile soil; Pythium—negative control germination of beans in the soil infected by P. ultimum; P1, 20, 59, 104, 125, 133 -germination of beans in soil infected by P. ultimum and Trichoderma (gray bars) or only Trichoderma (black bars).

FIG. 10 Protective effect of Trichoderma on bean seedlings growing in P. ultimum infective soil. Positive control (control)—growth of beans in the sterile soil; Pythium—negative control growth of beans in the soil infected by P. ultimum; P1, PZ1/06, PZ6/06, PZ19/06—growth of beans in soil infected by P. ultimum and Trichoderma (gray bars) or only Trichoderma (black bars) presented in the patent application P393021.

FIG. 11 Nucleotide (SEQ ID NO: 1) and amino acids (SEQ ID NO: 2) sequences of farnesyl pyrophosphate synthase from S. cerevisiae.

### DETAILED DESCRIPTION OF THE INVENTION

The mevalonate pathway mutated in the strains according to the invention is a place where dolichol and ergosterol are produced. Ergosterol is a compound of the cell membranes and secretory vesicles, organelles which are important for protein modification and secretion whereas, dolichol in the phosphorylated form is directly taking part in protein glycosylation. Furthermore, in the mevalonate pathway some antifungal and antibacterial metabolites such as terpenoids, trichodermin, harzianum A, mycotoxin T2, lignoren, ergokonin A and B and viridin are produced (Sivasithamparam and Ghisalberti 1998).

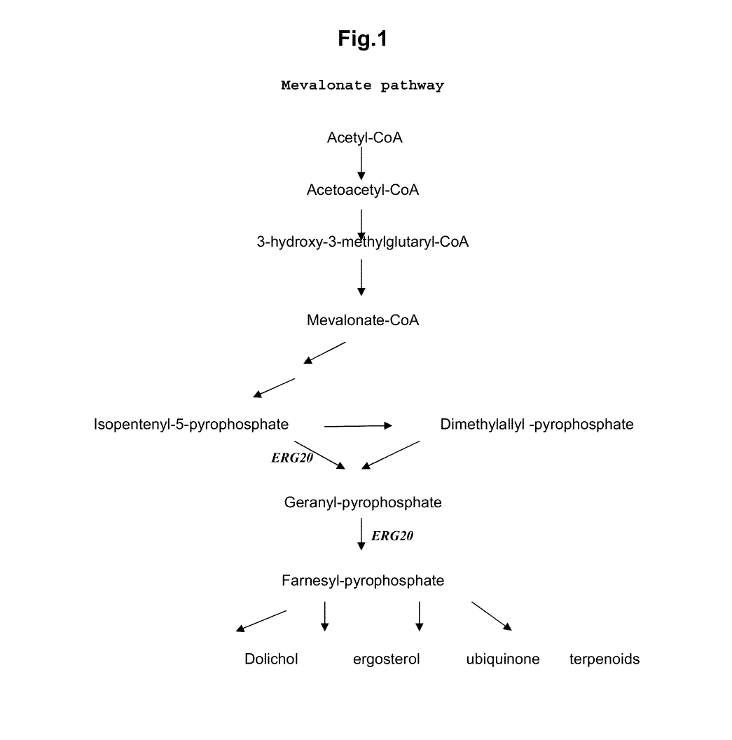

Farnesyl pyrophosphate is a substrate for formation of all these compounds and it is synthesized by farnesyl pyrophosphate synthase encoded by ERG20 gene. This enzyme has two activities, first synthesis of geranyl pyrophosphate and then transfer of the next isopentenyl group on the geranyl pyrophosphate to form farnesyl pyrophosphate. Farnesyl pyrophosphate is a substrate for all branches of the mevalonate pathway (FIG. 1).

According to the invention, increased antifungal and biocontrol activity of Trichoderma atroviride is achieved by increasing activity of farnesyl phyrophosphate synthase.

According to the invention, increase activity of farnesyl pyrophosphate synthase is achieved by overexpression of the S. cerevisiae ERG20 gene encoding farnesyl pyrophosphate synthase in T. atroviride P1.

According to the invention, ERG20 gene is randomly integrated into the T. atroviride genome using transformation method.

In the method, according to the invention, advantageously, before introduction of the gene into the Trichoderma genome, the ERG20 gene from Saccharomyces cerevisiae is amplified using PCR method and ERGBam-U (CGGGATCCATGGCTTCAGAA) (SEQ ID NO: 3) and

ERGBam-L (CGGGATCCCTATTTGCTTCTC)(SEQ ID NO: 4) primers. The PCR method enables a DNA fragment to be obtained with suitable ends necessary for insertion into the expression vector. The gene is cloned under the gpdA (glyceraldehyde-3-phosphate dehydrogenase) promoter and trpC (indole-3-glycerol-phosphate synthase) terminator into the pAN521N vector. The chosen promoter and terminator, profitably, originate from the filamentous fungus Aspergillus closely related to Trichoderma. To obtain expression, the gene should be cloned under the promoter easily recognized by the host. In this respect filamentous fungi are rather un-demanding, being able to recognize promoters from other, not so closely related fungi, for example yeast ones.

Profitably, the pAN521N vector is digested with BamHI restriction endonuclease, and the ERG20 PCR fragment is ligated using T₄ ligase. Next, the resulting vector is digested with NcoI restriction endonuclease, sticky ends are blunted using Mung Bean or S1 nucleases and selfligated.

Application of the pAN521N vector requires the use of BamHI restriction enzyme for digestion. The additional ATG has to be removed the way that, the vector has to be digested with NcoI and the obtained sticky ends have to be blunted by Mung Bean or S1 nucleases to avoid shifting of the Open Reading Frame of the ERG20 gene.

To obtain transformants a cotransformation method was used which means that a mixture of two vectors is used, one carrying the gene encoding the protein which we want to express and the second one, the helper vector, carrying marker gene. In this case, the first vector contains the ERG20 gene and the second one carries Hygromycin B resistance marker gene.

The two vectors, are used in a ratio of 10-15 parts of the first vector and one part of the helper one. It is known that a mixture of vectors used for transformation facilitates uptake of DNA into the cells of filamentous fungi.

Transformants are, selected on plates with a medium containing Hygromycin B and 0.1% Triton X-100. The selection medium should contain Hygromycin B to allow selection of transformants insensitive to Hygromycin B which will grow on this medium, whereas Triton X-100 prevents overgrowth of the plate and enables selection of individual transformants.

Next, transformants insensitive to Hygromycin B are checked for the presence of the ERG20 gene. To this end, DNA is obtained from the transformants and on the template of this DNA a DNA fragment is amplified using primers specific for the ERG20 gene. Next, the obtained PCR products are analyzed on an agarose gel and bands of about 1200 by are isolated and sequenced. Simultaneously, two control PCR reactions are performed: a positive control reaction on the template of S. cerevisiae genomic DNA, and a negative control using T. atroviride P1 (host strain—untransformed) DNA as the template. Sequences obtained from the sequencing analysis are compared with the known ERG20 nucleotide sequence.

DNA from the transformants could also be analyzed by the Southern blotting method, but such an analysis takes more time and is more expensive. When Southern blotting is used DNA is digested, with EcoRI, subjected to electrophoresis on agarose gel, denatured, transferred to a nylon membrane and hybridized with a radioactive probe synthesized on the template of the ERG20 gene (PCR product as above).

The Trichoderma genomic DNA should be digested with a restriction enzyme which does not cut inside the ERG20 gene, for example: BamHI, ClaI, HpaI, HpaII, NdeI, NotI, PvuI, PvuII, SalI, SmaI, SphI, XbaI.

Transformants carrying the ERG20 gene were also analyzed by the Northern blotting method. For this analysis total RNA was obtained and it was shown that the transformants contained an ERG20 transcript. The transformants obtained and verified in the described manner were named SGUL20/10, SGUL59/10, SGUL104/10, SGUL125/10 and SGUL133/10 and were used for further investigations.

Below the examples are provided on embodiments of the invention.

### EXAMPLE I

The ERG20 gene was amplified on the template of S. cerevisiae genomic DNA by PCR using ERGBam-U (CGGGATCCATGGCTTCAGAA) (SEQ ID NO: 3) and ERGBam-L (CGGGATCCCTATTTGCTTCTC) (SEQ ID NO: 4) primers.

The PCR method (Polymerase Chain Reaction) consists in amplification of a DNA fragment using oligonucleotide primers homologous to both ends of the DNA stretch to be amplified and using cyclic heating and cooling when primers are cyclically bound to the template DNA and a new nucleotide chain is synthesized between the primers. During the following temperature cycles the newly synthesized DNA becomes a template for further synthesis. The ERG20 gene obtained in this way was cloned under the gpdA (glyceraldehyde-3-phosphate dehydrogenase) promoter and trpC (indole-3-glycerol-phosphate synthase) terminator in the pAN521N vector (NCBI Accession number Z32697).

The vector was digested with BamHI restriction endonuclease, and the ERG20 PCR product was ligated into the vector using T₄ ligase (Promega). Next, the resulted vector was digested with NcoI restriction endonuclease and the sticky ends were blunted with Mung Bean nuclease (Promega) and the vector was selfligated. The resulting plasmid was used to transform T. atroviride P1 strain. The transformation was performed according to Kubicek-Pranz, E. M., Gruber, F., Kubicek, C. P. (1991) Transformation of Trichoderma reesei with the cellobiohydrolase II gene as a means for obtaining strains with increased cellulase production and specific activity. J. Biotechnol. 20, 83-94, and Mach, R. L., Schindler, M., Kubicek, C. P. (1994) Transformation of Trichoderma reesei based on Hygromycin B resistance using homologous expression signals. Curr. Genet. 25, 567-570. Transformants were selected on medium containing Hygromycin B and 0.1% Triton X-100.

Transformants growing in the presence of Hygromycin B were checked for the presence of ERG20 gene integrated into their genomic DNA. To this end, genomic DNA was obtained from the transformants using Wizard Genomic DNA Purification Kit (Promega). On the templates of the DNA from the transformants a fragment of DNA was amplified using primers ERG20Bam-U and ERG20Bam-L. Next, the PCR products were analyzed on agarose gel and bands of about 1200 bp were isolated and sequenced to confirm the presence of yeast ERG20 DNA in the genome of the transformants. In parallel, total RNA was isolated from the transformants and expression of the ERG20 gene was confirmed by dot-blot Northern analysis showing the presence of ERG20 mRNA. Transformants obtained and verified as above were named SGUL20/10, SGUL59/10, SGUL104/10, SGUL125/10 and SGUL133/10 and were used for further investigations.

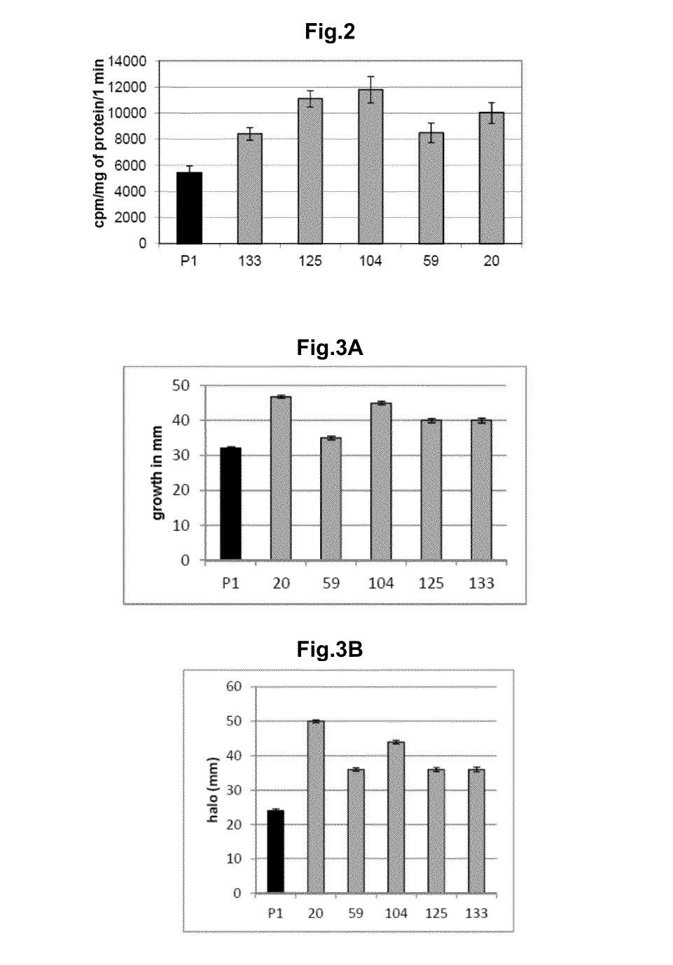

Overexpression of the ERG20 gene in T. atroviride obtained according to the innovation increased the activity of farnesyl pyrophosphate synthase (FIG. 2). Overexpression of the ERG20 gene in T. atroviride caused an increase of the farnesyl pyrophosphate synthase activity in the cell free extract obtained from all the transformants. The most pronounced increase, to 100% of the control farnesyl pyrophosphate synthase activity, was noticed for the SGUL104 (104) and SGUL125 (125) strains.

### EXAMPLE II

For simple screening of the hydrolytic properties of the obtained strains their growth was examined on plates with MM medium supplemented with carboxymethylcellulose as a carbon source. The area of growth was measured after three days of cultivation and compared with that of the control strain (FIG. 3A). All the transformants grew faster than the control strain suggesting that their higher hydrolytic activity enabled more efficient utilization of the complex carbon source.

Since hydrolytic enzymes are secreted to the medium, carboxymethylcellulose could be hydrolyzed and form halo surrounding the fungal colony. Size of the halo surrounding fungal colony correlates with the amount of secreted hydrolases (FIG. 3B).

### EXAMPLE III

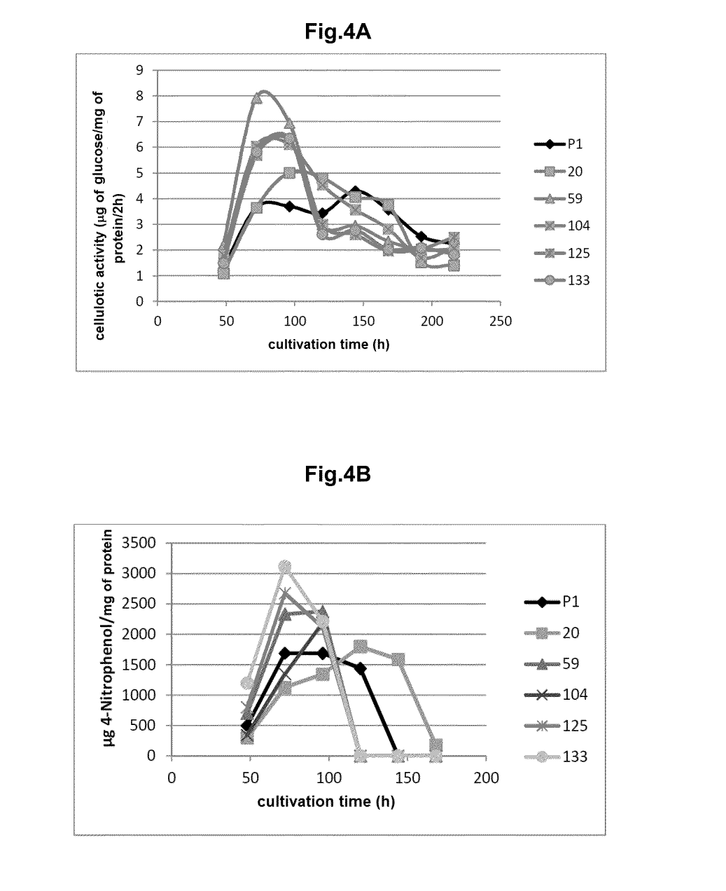

To check whether the transformants secreted higher amounts of cellulases or the cellulases were more active, at first the cellulolytic activity of proteins secreted to the cultivation medium was measured (FIG. 4A). At the same time, activity of N-acetyl-β-D-glucosaminidase was analyzed (FIG. 4B). It was shown that cellulases and N-acetyl-β-D-glucosaminidase secreted by the transformants (SGUL20/10, SGUL59/10, SGUL104/10, SGUL125/10, SGUL133/10) exhibited higher activity compared to the enzymes secreted by the control P1 strain. Trichoderma strains characterized with high chitinolytic activity could be used as bio-insecticides against crop-destroying insects. Since the outer skeleton of insects (cuticle) is built in large part with chitin more active hydrolysis of chitin may increase the effectiveness of bio-insecticide.

### EXAMPLE IV

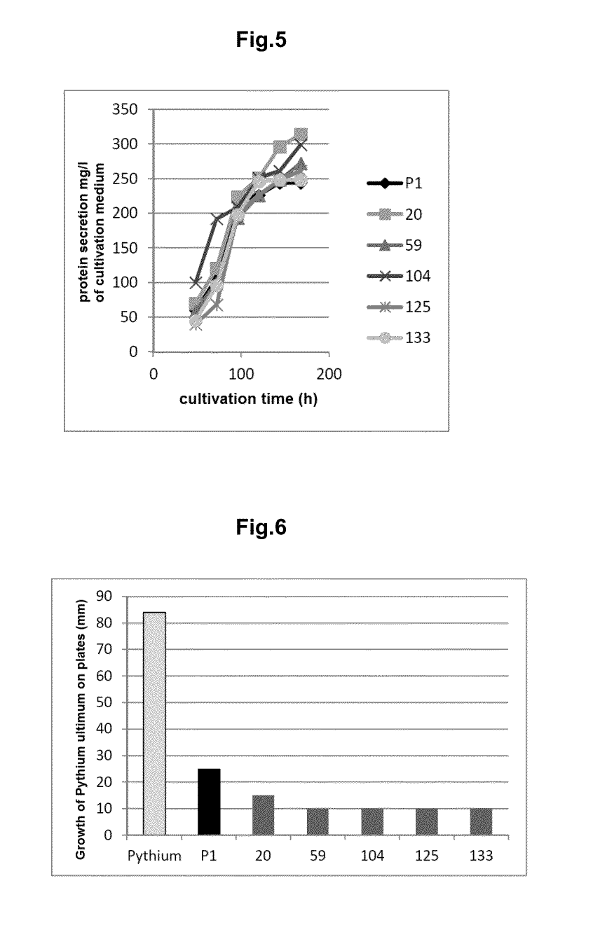

Next, the amount of proteins secreted to the cultivation medium was measured. The results obtained for transformants (SGUL20/10, SGUL59/10, SGUL104/10, SGUL125/10 and SGUL133/10) and for the control strain P1 are shown in FIG. 5. The amount of protein secreted by the transformants is up to 29% higher compared to the control strain P1.

### EXAMPLE V

To check the antifungal properties of the metabolites secreted by the transformants they and the control strain were cultivated on plates covered with cellophane. After the plates had been grown over, the cellophane with the Trichoderma was removed and the plates were used for cultivation of the plant pathogen P. ultimum. A negative control comprised plates not subjected to Trichoderma culturing. The growth of P. ultimum was strongly inhibited by the metabolites secreted by transformants compared to the control Trichoderma strain (FIG. 6).

### EXAMPLE VI

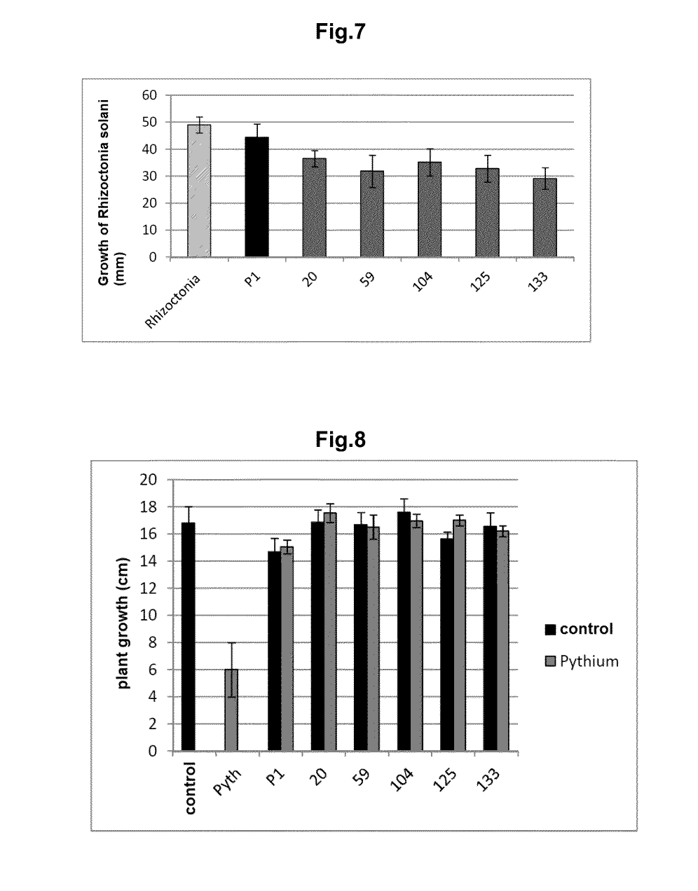

The mevalonate pathway is a place where a lot of volatile compounds are synthesized. It is known that some of them have antifungal activity. To analyze antifungal activity of volatile compounds synthesized by the transgenic strains plant pathogen Rhizoctonia solani was cultivated in the atmosphere of volatiles liberated by the transformants SGUL20/10, SGUL59/10, SGUL104/10, SGUL125/10 and SGUL133/10 and the control P1 strain (FIG. 7). It was found that volatile compounds secreted by the transformants more efficiently inhibited growth of the pathogen compared to the control P1 strain.

### EXAMPLE VII

Since the experiments described above revealed enhanced antifungal properties of the transformed Trichoderma strains proper biocontrol experiments were performed using the bean Phaseolus vulgaris L. Bean seeds (Phaseolus vulgaris L. (var. nanus L.)) were coated with 5×10⁸ spores of Trichoderma per 10 g of seeds. Pathogen-infected soil was prepared by adding 2.2 g of fungal biomass of Pythium ultimum to 1 l of sterile soil. Beans were allowed to germinate in pathogen-infected soil for ten days and the number and height of the seedlings was investigated.

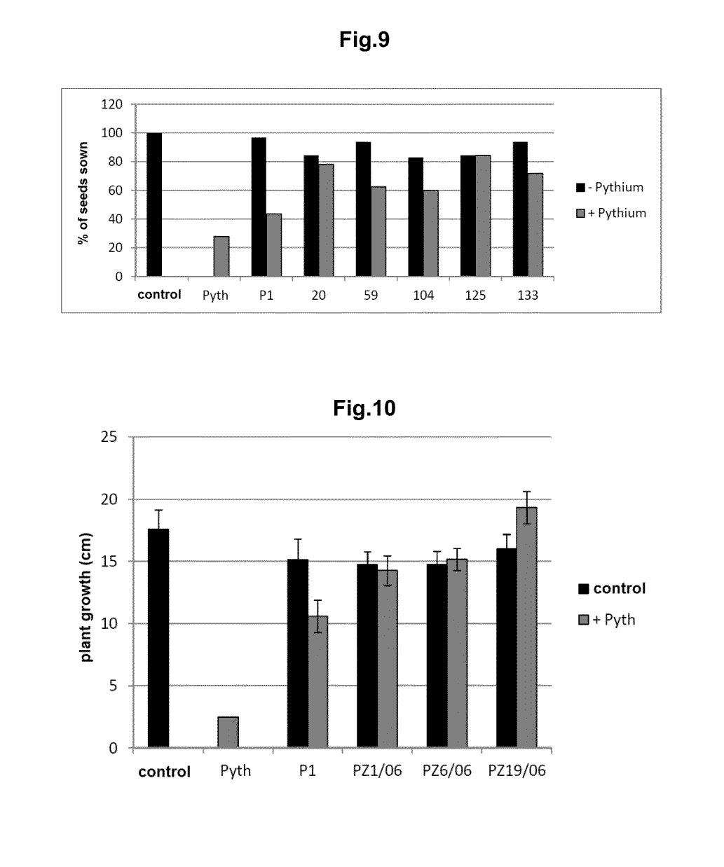

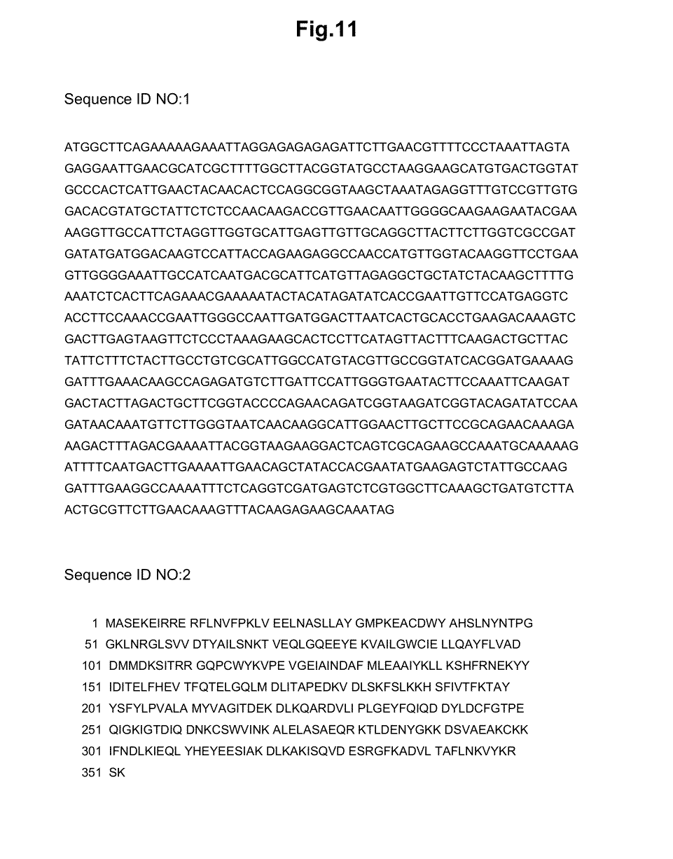

Infection by P. ultimum almost completely inhibited the growth of plants (FIG. 8). Plants are clearly protected from infection by all strains of Trichoderma transformants, moreover, in the presence of transformants beans grow better than protected by the parental P1 strain. In addition, transformed strains significantly raise the percentage of germinated seeds in the presence of pathogen compared to the control P1 strain (FIG. 9).

There was also a positive effect of the Trichoderma strains on plant growth promotion which was not observed for the strains covered by the application P393021. Strains of the present invention result in up to 20% higher plant growth compared to the control strain P1. In the presence of the pathogen (Pythium ultimum) growth of plants matched the growth of the control plants cultivated in the non-infected soil, and furthermore, in the presence of SGUL20/11, SGUL104/11 and SGUL125/11 exceeded the growth of the control plants what was observed only for one strain (FIG. 10) from the patent application No P393021.

Strains presented here, provide a comprehensive increase of production of all active compounds in the mevalonate pathway and, at the same time, increasing activity of the glycosylation process different way than previously known, for example in the application No P393021.

The resulting new strains of Trichoderma provide an increased activity of farnesyl pyrophosphate synthase, leading to increase activity of cellulases and N-acethyl-β-D-glucosidase secreted to the cultivation medium. Furthermore, the increased activity of farnesyl pyrophosphate synthase resulted in the increased activity of metabolites released to the medium. Also, the increased activity of farnesyl pyrophosphate synthase resulted in the increased antifungal activity of volatile compounds emitted during growth. Moreover, increase of farnesyl pyrophosphate synthase activity increases biocontrol activity against Pythium ultimum.

The most spectacular effect of this invention is an increased liberation of volatile compounds with antifungal activity, increased secretion of hydrolytic enzymes, increased cellulolytic activity, increased liberation of secondary metabolites by the transformants compared to the control P1 strain and a significant increase of biocontrol activity of the ERG20-transformed strains compared to the control P1 strain.

The improved antifungal effect is not connected with any additional costs for cultivation of the transformants and does not require any additional manipulations.

1. A mutated strain of Trichoderma spp. comprising a recombinant DNA containing an ERG20 gene coding for farnesyl pyrophosphate synthase.

2. The Trichoderma strain according to claim 1, wherein the Trichoderma strain is Trichoderma atroviride.

3. The Trichoderma strain according to claim 1, wherein the gene encoding farnesyl pyrophosphate synthase originates from Saccharomyces cerevisiae.

4. The Trichoderma strain according to claim 1, wherein the gene encoding farnesyl pyrophosphate synthase comprises SEQ ID NO.1.

5. The Trichoderma strain according to claim 4, wherein the farnesyl pyrophosphate synthase comprises an amino acid sequence that is at least 50%, at least 55%, at least 60%, at least 65%, at least 70%, at least 75%, at least 80%, at least 85%, at least 90%, at least 95% or 100% identity with SEQ ID NO:2.

6. The Trichoderma strain according to claim 3, wherein the ERG20 gene from S. cerevisiae is integrated into the genome of Trichoderma.

7. The Trichoderma strain according to claim 3, wherein the ERG20 gene from S. cerevisiae is integrated into the genome of Trichoderma, and the strain overexpress the farnesyl pyrophosphate synthase.

8. A method of obtaining the Trichoderma strain according to claim 1, comprising: transforming Trichoderma with a DNA molecule coding for farnesyl pyrophosphate synthase, wherein the DNA molecule coding for the farnesyl pyrophosphate synthase is overexpressed.

9. The method according to claim 8, wherein the DNA molecule coding for farnesyl pyrophosphate synthase originates from S. cerevisiae and is integrated into the genome of Trichoderma.

10. A method of controlling undesired organisms, comprising: contacting undesired organisms with the Trichoderma strain of claim 1, wherein the ERG20 gene encoding farnesyl pyrophosphate synthase is overexpressed.

11. The method according to claim 10, wherein the ERG20 gene encoding farnesyl pyrophosphate synthase originates from S. cerevisiae.

12. The method according to claim 10, wherein fungal diseases of plants and crops are treated.

13. The method according to claim 10, wherein Pythium ultimum is inhibited.

14. A method for increasing plant growth and/or crop yields, comprising: treating a plant or crop with the Trichoderma strain of claim 1, wherein the ERG20 gene encoding farnesyl pyrophosphate synthase is overexpressed.

15. A biocidal composition comprising the Trichoderma strain of claim 1, wherein the ERG20 gene encoding farnesyl pyrophosphate synthase is overexpressed.

16. The composition of claim 15, wherein the composition is useful for increasing the crop yields.


SEQUENCE LISTING (4 sequences)

SEQ ID NO: 1 - DNA, 1059 bp, Saccharomyces cerevisiae
atggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatag

SEQ ID NO: 2 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 3 - DNA, 20 bp, Artificial Sequence
  PCR primer
cgggatccatggcttcagaa

SEQ ID NO: 4 - DNA, 21 bp, Artificial Sequence
  PCR primer
cgggatccctatttgcttctc